(19) United States
(12) Patent Application Publication
(10) Pub. No.: US 2021/0055272 A1
(43) Pub. Date: Feb. 25, 2021

(54) PORTABLE ENVIRONMENTAL SENSOR MEASURING AND CORRECTING SYSTEM AND THE METHOD THEREOF

(71) Applicant: CENTER FOR INTEGRATED SMART SENSORS FOUNDATION, Daejeon (KR)

(72) Inventor: Hyun Tae CHO, Daejeon (KR)

(21) Appl. No.: 16/690,832

(22) Filed: Nov. 21, 2019

(30) Foreign Application Priority Data
Aug. 23, 2019 (KR) 10-2019-0103658
Aug. 23, 2019 (KR) 10-2019-0103659

(51) Int. Cl. G01N 33/00; G01N 15/02

(52) U.S. Cl. CPC G01N 33/0004; G01N 15/0205

(57) ABSTRACT

The present invention which relates to portable environmental sensor measuring and correcting system measuring harmful environmental pollution level by using an environmental sensor in which data is corrected and the method thereof, includes a system correcting unit for removing noise of an environmental sensor, correcting gradient difference according to measured sensing value based on air pollutant concentration value of the environmental sensor in which the noise is removed according to a correcting parameter, and correcting environmental sensor value according to temperature-humidity change which changes characteristics of the environmental sensor, a contamination measuring unit for displaying pollution status by measuring pollution level for the measured sensing value according to corrected value, and a baseline correcting unit for correcting error of the measured sensing value by using national measurement network data based on location information.

This application claims the priority benefit of Korean Patent Application Nos. 10-2019-0103658 filed on Aug. 23, 2019 and 10-2019-0103659 filed on Aug. 22, 2019, in the Korean Intellectual Property Office, the disclosure of which is incorporated herein by reference.

### BACKGROUND

### 1. Field of the Invention

The present invention relates to portable environmental sensor measuring and correcting system and the method thereof, more particularly, system for measuring harmful environmental pollution level by using an environmental sensor in which data is corrected and the method thereof.

### 2. Description of Related Art

As various air pollutants are released into the atmosphere due to population growth caused by industrial development and urbanization, the air pollution problem in the area where we live is serious.

The various air pollutants, which are very small and invisible substances, may be released from many indoor activities of people, finishing materials used in interior architecture, household items, and the like.

However, since an existing environmental sensor for measuring air pollutant determines a sensing value measured based on a preset value, there is a limit that it may not follow different air pollutant concentration standards by region and location and there are frequent measurement errors according to weather conditions and hours of use.

### PRIOR ART REFERENCE

### Patent Documents

Korean Patent Publication No. 10-2008-0018452 (Feb. 28, 2008, Publication)

### SUMMARY

The purpose of the present invention is to improve measurement accuracy of an environmental sensor according to error correction by correcting air pollutant concentration value according to baseline in which noise of the environmental sensor is removed and temperature-humidity change which changes characteristics of the environmental sensor.

According to at least one example of embodiments, an environmental sensor measuring and correcting system includes a system correcting unit for removing noise of an environmental sensor, correcting gradient difference according to measured sensing value based on air pollutant concentration value of the environmental sensor in which the noise is removed according to a correcting parameter, and correcting environmental sensor value according to temperature-humidity change which changes characteristics of the environmental sensor, a contamination measuring unit for displaying pollution status by measuring pollution level for the measured sensing value according to corrected value, and a baseline correcting unit for correcting error of the measured sensing value by using national measurement network data based on location information.

The environmental sensor may be a fine dust sensor, a VOC (Volatile Organic Compound) sensor, or a gas sensor.

The system correcting unit may acquire baseline by removing noise of the environmental sensor by applying a noise removing filter in the initial stage, and adjust the baseline by adjusting the correcting parameter according to gradient difference of the environmental sensor acquired in a first environment and a second environment having different measurement concentrations.

The system correcting unit may correct gradient difference according to the measured sensing value based on the adjusted baseline according to the correcting parameter on the system.

The system correcting unit may correct temperature-humidity value of the environmental sensor by using the measured sensing value, difference value of reference temperature and the current temperature, and difference value of reference humidity and the current humidity.

Also, according to at least one example of embodiments, the environmental sensor measuring and correcting system may further include a parameter processing unit for updating the correcting parameter by using environmental distribution information in the current location and region according to the location information.

The parameter processing unit may receive and update the correcting parameter for the environmental distribution information in the current location and region verified in an external server by the location information acquired through interlocked mobile device from the mobile device, and adjust the baseline of the environmental sensor according to the updated correcting parameter.

The baseline correcting unit may correct error of the measured sensing value by adjusting the baseline of the environmental sensor according to the national measurement network data according to the location information acquired through interlocked mobile device.

The baseline correcting unit may correct error of the measured sensing value by adjusting the baseline of the environmental sensor by drift caused by hours of use.

According to at least one example of embodiments, an environmental sensor measuring and correcting system may include a system correcting unit for removing noise of a fine dust sensor, correcting gradient difference according to measured fine dust concentration value based on fine dust concentration value of the fine dust sensor in which the noise is removed according to a correcting parameter, and correcting fine dust sensor value according to temperature-humidity change which changes characteristics of fine dust, and a contamination measuring unit for displaying pollution status by measuring pollution level for the measured fine dust concentration value according to corrected value.

The system correcting unit may acquire baseline by removing noise of the fine dust sensor by applying a noise removing filter in the initial stage, and adjust the baseline by adjusting the correcting parameter according to gradient value according to fine dust concentration acquired in a first environment and a second environment having different fine dust concentrations.

The system correcting unit may correct gradient difference according to the measured fine dust concentration value based on the adjusted baseline according to the correcting parameter on the system.

The system correcting unit may correct temperature-humidity value of the fine dust sensor by using the measured fine dust concentration value, difference value of reference temperature and the current temperature, and difference value of reference humidity and the current humidity.

Also, according to at least one example of embodiments, the environmental sensor measuring and correcting system may further include a parameter processing unit for updating the correcting parameter by using fine dust distribution information in the current location and region according to location information.

The parameter processing unit may receive and update the correcting parameter for the fine dust distribution information in the current location and region verified in an external server by the location information acquired through interlocked mobile device from the mobile device, and adjust the baseline of the fine dust sensor according to the updated correcting parameter.

Also, according to at least one example of embodiments, the environmental sensor measuring and correcting system may further include an aging correcting unit for correcting error of the measured fine dust concentration value caused by hours of use.

The aging correcting unit may correct error of the measured fine dust concentration value by adjusting the baseline of the fine dust sensor by drift caused by hours of use.

According to at least one example of embodiments, an environmental sensor measuring and correcting method include removing noise of an environmental sensor, correcting gradient difference according to measured sensing value based on all pollutant concentration value of the environmental sensor in which the noise is removed according to a correcting parameter, correcting environmental sensor value according to temperature-humidity change which changes characteristics of the environmental sensor, displaying pollution status by measuring pollution level for the measured sensing value according to corrected value, and correcting error of the measured sensing value by using national measurement network data according to location information.

Also, according to at least one example of embodiments, the environmental sensor measuring and correcting method may further include updating the correcting parameter by using environmental distribution information in the current location and region according to location information.

The correcting error of the measured sensing value may correct error of the measured sensing value by adjusting the baseline of the environmental sensor by drift caused by hours of use.

According to at least one example of embodiments, an environmental sensor measuring and correcting method may include removing noise of a fine dust sensor, correcting gradient difference according to measured fine dust concentration value based on fine dust concentration value of the fine dust sensor in which the noise is removed according to a correcting parameter, correcting fine dust sensor value according to temperature-humidity change which changes characteristics of fine dust, and displaying pollution status by measuring pollution level for the measured fine dust concentration value according to corrected value.

Also, according to at least one example of embodiments, the environmental sensor measuring and correcting method may further include updating the correcting parameter by using fine dust distribution information in the current location and region according to location information.

Also, according to at least one example of embodiments, the environmental sensor measuring and correcting method may further include error of the measured fine dust concentration value caused by hours of use.

According to example embodiments, measurement accuracy of an environmental sensor may be improved according to error correction by correcting baseline in which noise of the environmental sensor is removed and air pollutant concentration value according to temperature-humidity change which changes characteristic of the environmental sensor.

Also, according to example embodiments, error caused when measuring air pollutant in each region may be minimized by updating a correcting parameter for air pollutant distribution information in the current location and region according to location information acquired through interlocked mobile device.

Also, according to example embodiments, error of air pollutant concentration value may be minimized by adjusting baseline of an environmental sensor according to hours of use.

### BRIEF DESCRIPTION OF THE DRAWINGS

These and/or other aspects, features, and advantages of the present invention will become apparent and more readily appreciated from the following description of embodiments, taken in conjunction with the accompanying drawings of which:

FIG. 1 is a block diagram illustrating detailed configuration of an environmental sensor measuring and correcting system according to an example of embodiments;

FIGS. 2A to 2C illustrate examples of structure of an environmental sensor measuring and correcting system according to an example of embodiments;

FIG. 3 is a flow chart illustrating an operation method of an environmental measuring and correcting system according to an example of embodiments;

FIGS. 4 to 15 illustrate experimental graphs according to an example of embodiments;

FIG. 16 illustrates an example of structure of an environmental sensor measuring and correcting system according to an example of embodiments;

FIG. 17 is a flow chart illustrating an operation method of an environmental sensor measuring and correcting system according to an example of embodiments;

FIGS. 18 and 19 are drawings for illustrating example of updating a correcting parameter according to an example of embodiments;

FIG. 20 is a drawing for illustrating an example of correcting baseline by using national measurement network data according to an example of embodiments;

FIG. 21 is a flow chart illustrating an operation method of an environmental sensor measuring and correcting method according to an example of embodiments;

FIG. 22 is a block diagram illustrating detailed configuration of an environmental sensor measuring and correcting system according to an example of embodiments;

FIG. 23 is a flow chart illustrating an operation method of an environmental sensor measuring and correcting system according to an example of embodiments; and

FIG. 24 is a flow chart illustrating an operation of an environmental sensor measuring and correcting method according to an example of embodiments.

### DETAILED DESCRIPTION

Hereinafter, some example embodiments will be described in detail with reference to the accompanying drawings. Regarding the reference numerals assigned to the elements in the drawings, it should be noted that the same elements will be designated by the same reference numerals, wherever possible, even though they are shown in different drawings.

Also, terminologies used herein refer to terms used to appropriately represent the example embodiments and may vary based on a reader, the intent of an operator, or custom of a field to which this disclosure belongs, and the like. Accordingly, the definition of the terms should be made based on the overall description of the present specification.

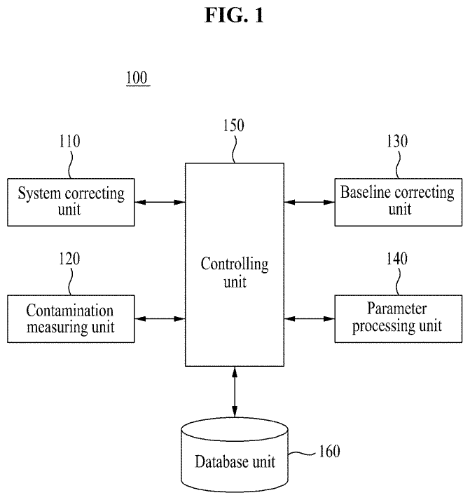

FIG. 1 is a block diagram illustrating detailed configuration of an environmental sensor measuring and correcting system according to an example of embodiments.

Referring to FIG. 1, an environmental measuring and correcting system according to an example of embodiments measures harmful environmental pollution level by using an environmental sensor in which data is corrected.

For this, an environmental measuring and correcting system according to an example of embodiment 100 may include a system correcting unit 110, a contamination measuring unit 120, and a baseline correcting unit 130, and may further include a parameter processing unit 140, a control unit 150, and a database unit 160.

The environmental measuring and correcting system 100, which is to measure more accurate air pollutant pollution level by correcting data of an environmental sensor, may be a form that the environmental sensor is included in structure of the system. Here, the environmental sensor may be a fine dust sensor, a VOC (Volatile Organic Compound) sensor, or a gas sensor, and air pollutant may refer to any substance detected from the environmental sensor.

The system correcting unit 110 removes noise of the environmental sensor, corrects gradient difference according to measured sensing value based on air pollutant concentration value of the environmental sensor in which the noise is removed according to a correcting parameter, and corrects environmental sensor value according to temperature-humidity change which changes characteristics of the environmental sensor.

In the initial stage of the system, the system correcting unit 110 may acquire baseline by removing noise of the environmental sensor by applying a noise removing filter, and adjust the baseline by adjusting the correcting parameter according to gradient difference of the environmental sensor acquired in a first environment and a second environment having different measurement concentrations.

For example, the system correcting unit 110 may acquire baseline corresponding to a corresponding environmental sensor after removing noise for the environmental sensor by using a noise removing filter. Since environmental sensors have different initial baseline values which are recognized for each sensor, the system correcting unit 110 may first perform acquiring baseline for the corresponding environmental sensor in the system.

Furthermore, since environmental sensors have different base lines, they should be corrected in an environment free of air pollutant. For example, the system correcting unit 110 may measure fine dust concentration value in each of a first environment where fine dust is 0 ug and a second environment where fine dust is about 50 ug, infer offset difference which varies according to fine dust concentration by adjusting gradient difference according to fine dust concentration value, and because of this, adjust baseline by adjusting the correcting parameter of the environmental sensor.

In the next stage of the system, the system correcting unit 110 may correct gradient difference according to measured sensing value based on baseline adjusted according to the correcting parameter on the system.

The environmental sensor measuring and correcting system 100 acquires sensing value from the environmental sensor. At this time, the acquiring method may measure output voltage in case of analog output and convert it to concentration, and it may use the acquired value as it is in case of digital output. Afterwards, the present invention performs a process for removing noise, and the process for removing noise may be performed by applying a noise removing filter as the same with the work performed in the initial stage of the system.

Accordingly, the system correcting unit 110 may correct gradient difference according to measured sensing value based on baseline by using below [Equation 1] in order to correct error according to concentration based on air pollutant concentration value of the environmental sensor in which the noise is removed.

ρ(x)=(x+αD·ΔD)+b_{baseline} [Equation 1]

Here, x indicates sensing value of the current actual measured air pollutant, αD indicates the current air pollutant concentration (measured concentration−reference concentration (0 ug)), and ΔD indicates offset value from actual air pollutant concentration. Also, when the actual air pollutant concentration is 0, b_{baseline} indicates measured offset baseline.

Afterwards, the system correcting unit 110 may correct temperature-humidity value of the environmental sensor by using the measured sensing value, difference value of reference temperature and the current temperature, and difference value of reference humidity and the current humidity.

When measuring air pollutant, temperature and humidity value may have an effect on air pollutant concentration value. Since change on air pollutant concentration value is caused by temperature-humidity value and this causes error in the measurement, correction of temperature-humidity is needed when measuring air pollutant. Accordingly, the system correcting unit 110 may correct temperature-humidity value of the environmental sensor by using difference value of reference temperature and the current temperature and difference value of reference humidity and the current humidity through below [Equation 2] and [Equation 3].

ρ(x)′=(ρ(x)+αT·ΔT) [Equation 2]

dp(x)_{corrected}=(ρ(x)′+αT·ΔT) [Equation 3]

Here, x indicates sensing value of the current actual measured air pollutant, ρ(x) indicates a value that the concentration value is corrected from the actual measured value, and ρ(x)′ indicates a value that the temperature value is corrected again.

Also, αT indicates the reference temperature−the current temperature value, and ΔT indicates the offset value (0.5 ug per 1 degree change). ΔT this time, the offset value may be changed according to characteristics of optical source and light receiving element.

Also, βH indicates the reference humidity−the current humidity value, and ΔH indicates the offset value (0.1 ug per 1% RH). At this time, the offset value may be changed according to characteristics of optical source and light receiving element.

The contamination measuring unit 120 displays pollution status by measuring pollution level for the measured sensing value according to corrected value.

For example, the contamination measuring unit 120 may classify pollution level into about 5 steps according to the measured sensing value, and by measuring pollution level through step classification from low risk to high risk, pollution status of the current air pollutant may be displayed through at least one of LED, display, and output sound.

The baseline correcting unit 130 corrects error of the measured sensing value by using national measurement network data according to location information.

The baseline correcting unit 130 may correct error of the measured sensing value by adjusting baseline of the environmental sensor according to national measurement network data according to location information acquired through interlocked mobile device.

The environmental sensor measuring and correcting system 100 may further include a communication module (not illustrated), and may interlock with a mobile device that a user has through the communication module. At this time, the mobile device may be at least one device of a smartphone, a desktop, a PC, a mobile terminal, a PDA, a laptop, a tablet PC, and a wearable device, and may be installed with an application for interworking with the environmental sensor measuring and correcting system 100. Furthermore, the mobile device may receive selection input of a user, and since it may include a display in a form of a touch screen which may perform a predetermined set of functions through a screen including touch-sensing area or may be a device including at least one physical button or virtual button, the kinds and forms are not limited thereto.

Accordingly, the environmental sensor measuring and correcting system 100 may receive location information in real-time through the mobile device, and may receive data needed to update baseline from near national measurement network based on location information. Accordingly, the baseline correcting unit 130 may correct error of measured sensing value by updating baseline received from national measurement network.

For example, when the fine dust concentration sensed in the environmental sensor measuring and correcting system 100 is 55 ug and data of near national measurement network device indicates 50 ug, 5 ug error occurs. This error is determined as error for baseline, and the baseline correcting unit 130 may correct the error to baseline offset. However, in this case, if only one-time or short time data is collected, error in measurement may be included, so it is desirable to collect a lot of data, find optimal value, and then correct the offset.

Also, the baseline correcting unit 130 may correct error of the measured sensing value by adjusting baseline of the environmental sensor by drift caused by hours of use.

In case of a fine dust sensor or a semiconductor gas sensor, aging occurs over time, and performance of the environmental sensor is degraded due to the material applied to the sensor. Accordingly, since drift occurs over time and causes continuous error in the measurement, this needs to be corrected. At this time, baseline correction is needed rather than whole correction.

Therefore, the baseline correcting unit 130 may correct error of measured sensing value by adjusting baseline of the environmental sensor by drift caused by hours of use. For example, the baseline correcting unit 130 may correct offset value so that baseline value of the environmental sensor in an environment where fine dust is 0 ug is 0 ug.

The parameter processing unit 140 may update the correcting parameter by using environmental distribution information in the current location and region according to location information.

The parameter processing unit 140 may receive and update the correcting parameter for environmental distribution information in the current location and region verified in an external server by location information acquired through interlocked mobile device from the mobile device, and adjust baseline of the environmental sensor according to the updated correcting parameter.

For example, when a user A living in Seoul moves to Busan and measures air pollutant concentration, since the user A measures with an environmental sensor corrected in Seoul, error may occurs according to difference in distribution of air pollutant in Seoul and distribution of air pollutant in Busan. Accordingly, the parameter processing unit 140 may adjust baseline of the environmental sensor by updating the correcting parameter for air pollutant distribution information for Busan.

The control unit 150 may control operations of the system correcting unit 110, the contamination measuring unit 120, the baseline correcting unit 130, and the parameter processing unit 140 of the environmental sensor measuring and correcting system 100, and may store data occurred in each component in the database unit 160.

The database unit 160 may store baseline value of the environmental sensor corrected in the processing for correcting environmental sensor, and may store and maintain gradient value according to the sensing value measured with the environmental sensor and correction value for the measured temperature and humidity.

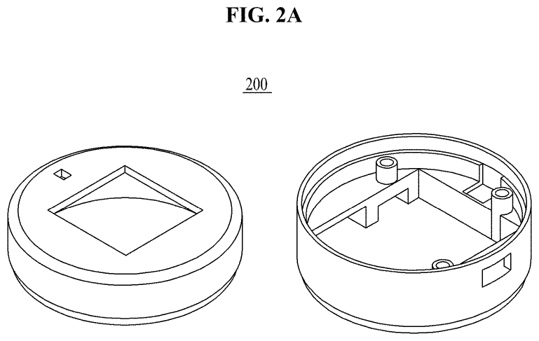

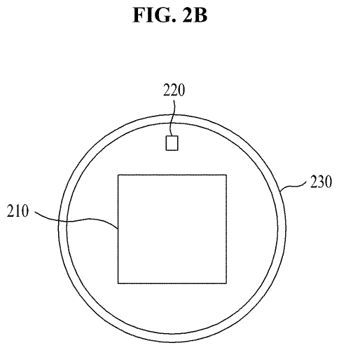

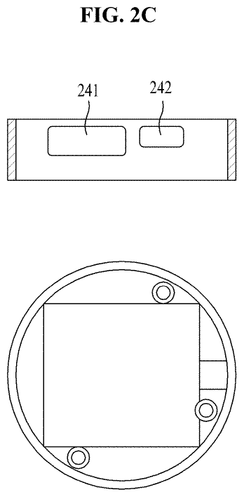

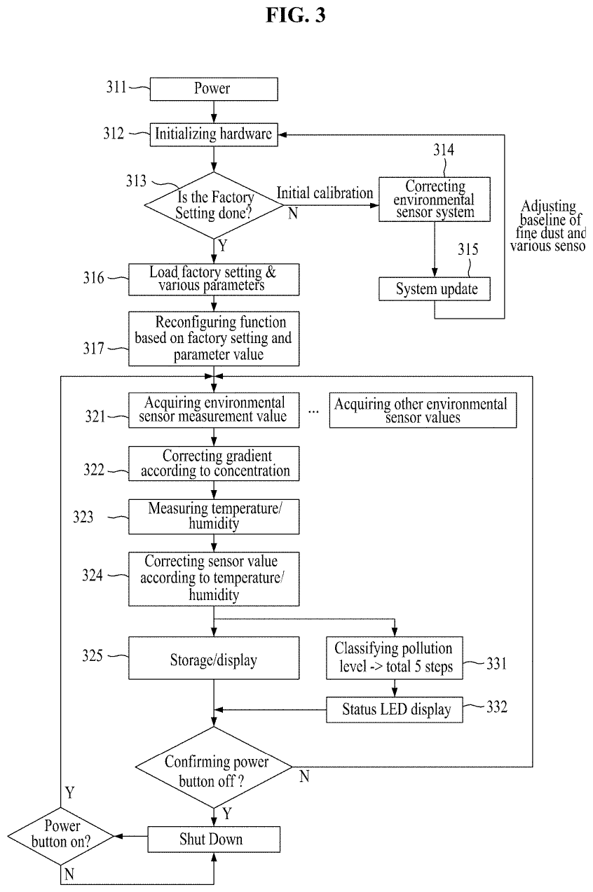

FIGS. 2A to 2C illustrate examples of structure of an environmental sensor measuring and correcting system according to an example of embodiments.

FIG. 2A illustrates a perspective view of an environmental sensor measuring and correcting system according to an example of embodiments, FIG. 2B illustrates a plane view of an environmental sensor measuring and correcting system according to an example of embodiments, and FIG. 2C illustrates a side view of an environmental sensor measuring and correcting system according to an example of embodiments.

As illustrated in FIG. 2A, an environmental sensor measuring and correcting system according to an example of embodiments 100 may be represented in a circular structure.

The environmental sensor measuring and correcting system 200 may mount at least one of a fine dust sensor measuring amount of fine dust, a VOC (Volatile Organic Compound) sensor for environmental measurement, and a gas sensor, and the sensor may be single or multiple. Also, it may include a temperature-humidity sensor, and the temperature-humidity sensor is used for measuring actual temperature and humidity, but the temperature-humidity data is used for correcting data of the fine dust sensor, the VOC sensor or the gas sensor.

The environmental sensor measuring and correcting system 200 may mount Bluetooth or WiFi communication module, and may transmit and receive data with a mobile device or connect to the Internet through communication module.

Also, the environmental sensor measuring and correcting system 200 may receive power supply through a battery, charge the battery through USB, and charge wirelessly by adding selectively coil. Accordingly, the environmental sensor measuring and correcting system 200 may display remaining battery measured through a battery monitor in a display 210 or transmit it to a mobile device or a server.

Referring to FIGS. 2B and 2C, the environmental sensor measuring and correcting system 200 includes a display window 210 indicating pollution level for measured air pollutant concentration value, and may include a temperature-humidity measuring vent 220 which is a vent that may measure temperature-humidity and a button 230. Also, the environmental sensor measuring and correcting system 200 may further include an outlet 241 and an inlet 242 which are located at the top and for input of air pollutant.

The display 210 is located at the top of the vent of apparatus and may be i.5 inches OLED. The environmental sensor measuring and correcting system 200 may display data measured through the display 210 and other necessary information, and classify and display pollution level for fine dust or surrounding air pollution into 5 steps (e.g., very bad, bad, normal, good, very good, and the like) through the display 210 and RGB LED.

The button 230 may convert mode from low power mode to measuring mode of the environmental sensor measuring and correcting system 200, and turn on/off the power.

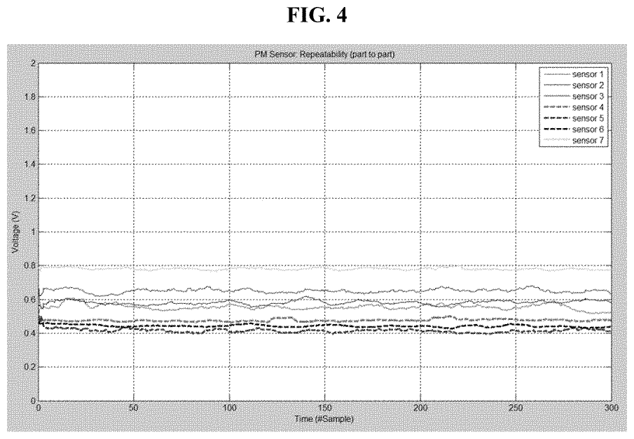

FIG. 3 is a flow chart illustrating an operation method of an environmental measuring and correcting system according to an example of embodiments, and FIGS. 4 to 13 illustrate experimental graphs according to an example of embodiments.

Referring to FIG. 3, when initial power is applied, an environmental sensor measuring and correcting system according to an example of embodiments confirms factory setting (or a correcting parameter) according to hardware initialization, and confirms whether correction of environmental sensors are performed (Steps 311 to 313). The process of Steps 311 to 313 is performed in only initial stage that power is applied to the environmental sensor measuring and correcting system.

A fine dust sensor, a VOC sensor or a gas sensor have different initial baseline value recognized for each sensor. For example, when find dust is 10 ug/m³, a first fine dust sensor may measure fine dust concentration as 15 ug, and a second fine dust sensor may measure and display fine dust concentration as Bug. Accordingly, the environmental sensor measuring and correcting system measures and corrects baseline of each different environmental sensor through Steps 311 to 317, and stores it in a system memory (or a database unit).

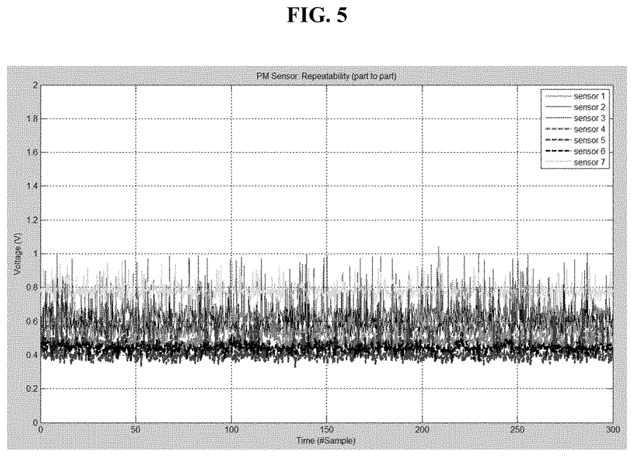

A graph illustrated in FIG. 4 indicates a result value when measuring through 7 fine dust sensors and removing noise, and it may be confirmed that they represent all different baselines. Also, a graph illustrated in FIG. 5 indicates a measurement value measured through 7 fine dust sensors, and it may be confirmed that a lot of noise is included in the measured fine dust concentration value.

Accordingly, the environmental sensor measuring and correcting system may correct baseline by removing noise of the environmental sensor in Steps 314 and 315.

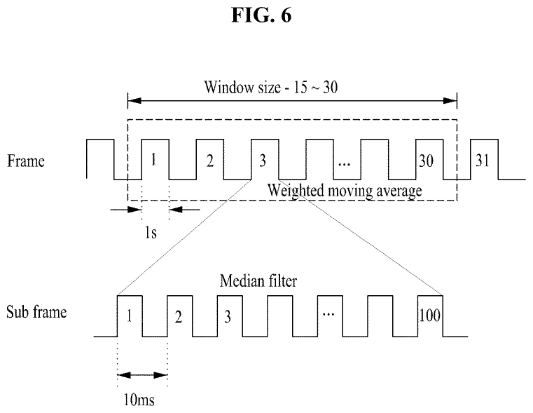

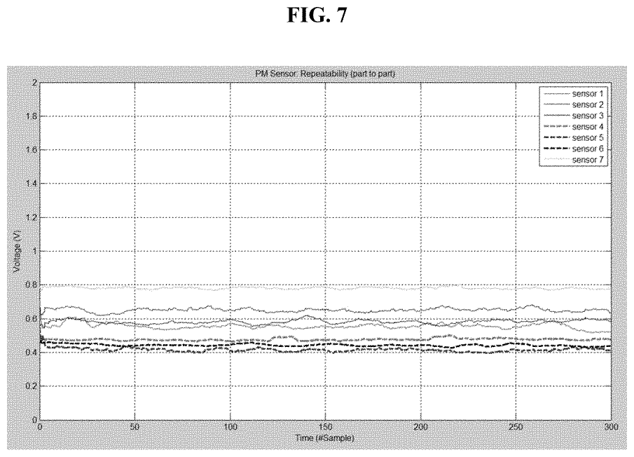

The environmental sensor measuring and correcting system may remove noise of the environmental sensor by using a noise removing filter of FIG. 6 and [Equation 4]. A result in FIG. 6 is displayed at every one second, and this value uses weighted moving average of whole frame. Also, window size is determined between 15 to 30, and may be applied differently depending on the environment to be measured.

$${\overline{x}}_{i} = ( {x}_{i} * α ) + ( \frac{1}{n - 1} {\sum }_{j = 0}^{i - 1} {x}_{j} ) * ( 1 - α ) \qquad [ Equation 4 ]$$

Here, xi indicates the current measurement value, and xj indicates average of the previous measurement value. The present invention may apply a low pass filter for controlling weighted value of the current measurement value and the previous measurement value through α. At this time, the measurement value at every one second unit is determined by sub-frame, and uses median value.

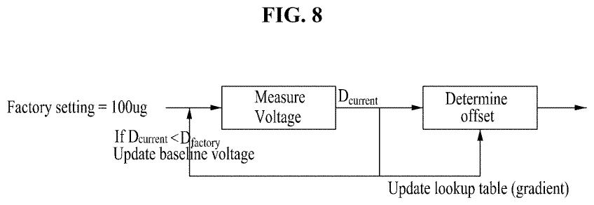

When the noise removing filter is applied, the environmental sensor measuring and correcting system may extract signal as illustrated in FIG. 7. However, in FIG. 7, since each of 7 fine dust sensors has each different baseline, this should be corrected in an environment having no fine dust.

Accordingly, in Steps 314 and 315, the environmental sensor measuring and correcting system acquires concentration value of the environmental sensor in an environment in which atmosphere pollutants are zero, records it in a memory, and may update baseline of the environmental sensor corrected in the environment having no atmosphere pollutant. For example, when fine dust concentration is measured as 5 ug in an environment in which fine dust is 0 ug, since offset is 5 ug, subtract this value on every measurement.

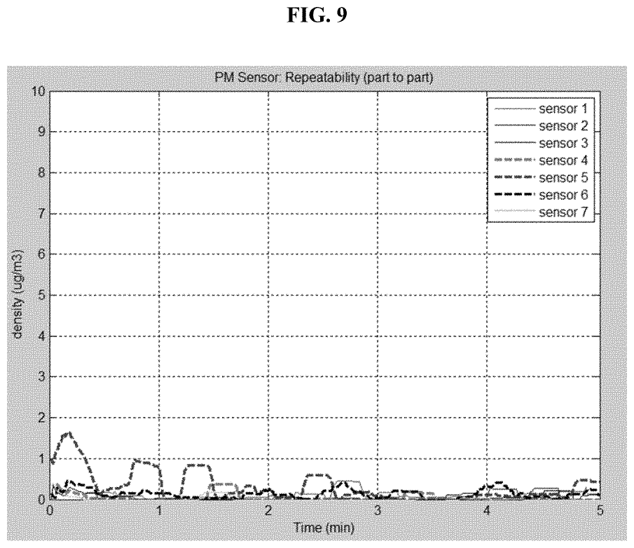

Referring to FIGS. 8 and 9, since 7 fine dust sensors have each different baseline, error according to the actual measured fine dust concentration occurs. In other words, gradient value according to the fine dust concentration may be changed. For example, if it is supposed that it is normal that in an environment in which fine dust is 0 ug, a first fine dust sensor measures 5 ug of fine dust concentration, and in an environment in which fine dust 50 ug, the first find dust sensor measures 55 ug of fine dust concentration, in case that a second fine dust sensor measures 10 ug of fine dust concentration in the environment in which fine dust is 50 ug, the second fine dust sensor measures 55 ug or 56 ug of fine dust concentration, not 60 ug of fine dust concentration in the environment in which fine dust is 50 ug.

This is by photodiode (light receiving unit) of the fine dust sensor and analogue circuit, and when the circuit of the fine dust sensor works with 3V, signal may be acquired by 3V output. In other words, this is a problem which occurs as upper boundary is fixed and lower boundary is variable.

For this, the environmental sensor measuring and correcting system should perform correction according to atmosphere pollutant concentration including fine dust concentration, and this may be corrected by acquiring and adjusting gradient value in a linear function.

In initial factory calibration, the environmental sensor measuring and correcting system may be input with air pollutant concentration value acquired through at least two environmental sensors. For example, the environmental sensor measuring and correcting system may acquire air pollutant concentration value in an environment in which air pollutant concentration is 0 (zero), and obtain gradient value by acquiring second air pollutant concentration value in an environment in which air pollutant concentration is 100. Accordingly, it is possible to infer offset difference which changes according to air pollutant concentration.

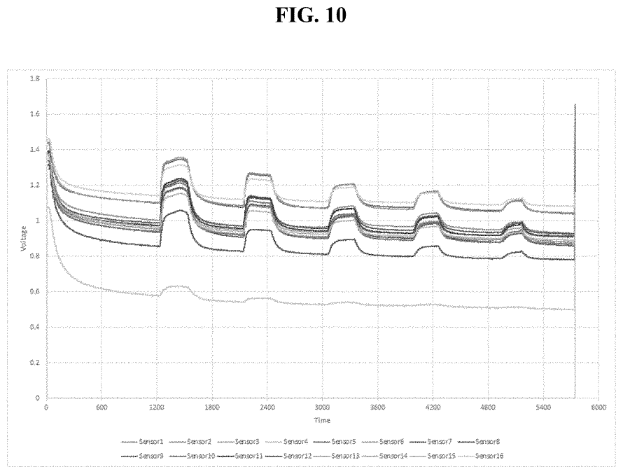

The fine dust sensor is described above with an example, but when a VOC sensor which is a semiconductor gas sensor is described with an example, FIG. 10 illustrates signal output value of 16 semiconductor VOC sensors as a result graph.

Referring to FIG. 10, it may be confirmed that all VOC sensors differently respond from different baselines. In case of the semiconductor gas sensor, the baseline may be acquired with the same method. However, in case of the semiconductor gas sensor, since heating time or warming up time for the sensor is needed unlike the fine dust sensor, the baseline is acquired after predetermined time. This time may be confirmed with a section in which resistance value rapidly changes and does not change as shown in the graph illustrated in FIG. 10, and the present invention acquires data in the confirmed section and stores it in a memory.

Also, when acquiring data after the next booting, the environmental sensor measuring and correcting system stores warming up time with data in order to use.

Furthermore, the environmental sensor measuring and correcting system may correct gradient value according to air pollutant concentration acquired with the semiconductor VOC sensor by proceeding with the same method as the fine dust sensor described above.

In the process for correcting the environment sensor, baseline value of the semiconductor gas sensor is stored, and heating time or warming up time for the semiconductor gas sensor is calculated and stored. Afterwards, when the system is on, normal data may be extracted after the heating time or warming up time.

In this process, the gradient value according to the concentration of the semiconductor gas sensor is stored in a memory or a database unit, and data for measured temperature-humidity may be also stored.

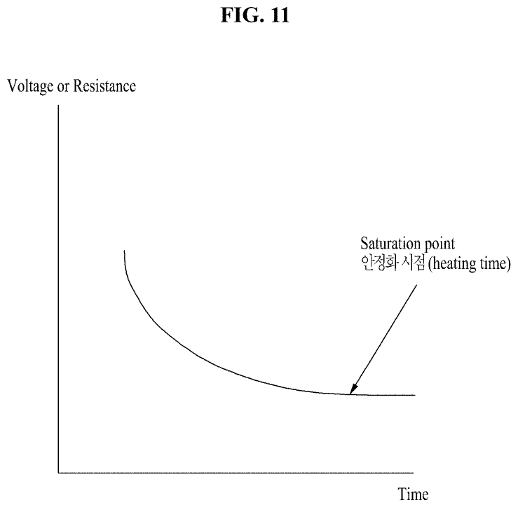

Also, in Step 314, the environmental sensor measuring and correcting system may measure and record heating time or warming up time of the gas sensor. As illustrated in FIG. 11, the heating time or warming up time should be performed again after the system enters idle or sleep mode to minimize power and wakes-up. In other words, when the environmental sensor measuring and correcting system restarts, heating time or warming up time of the semiconductor sensor is needed.

Accordingly, when the heating time or warming up time is not known, since every reset requires a long time for stabilizing, it is measured once at the time of initial correction and stored and after this, when the corresponding time comes, gas sensing may be performed. Accordingly, before measuring gas concentration with semiconductor type such as VOC and formaldehyde, heating time or warming up time is necessary.

Also, in the environmental sensor measuring and correcting system, when measuring by using the gas sensor, design for air circulation in the apparatus is important. For quick measuring, gas quickly flows in the system according to an example of embodiments located in the gas apparatus, and for accurate measuring, the inflow gas should quickly escape.

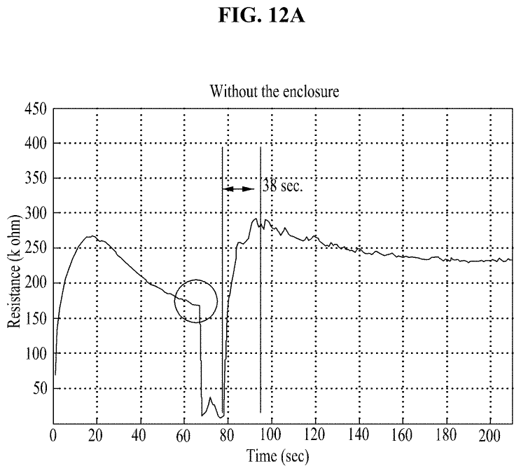

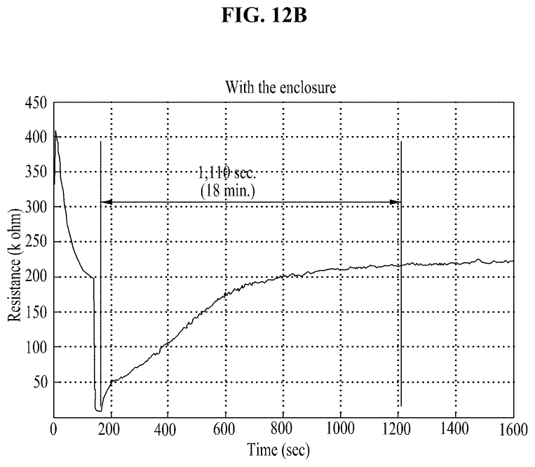

FIG. 12A illustrates recovery time of a sensor when apparatus design is good, the sensor protrudes outward, so the air circulation is good. As illustrated in FIG. 12A, it may be confirmed that the semiconductor sensor returns to stabilization time in approximately 38 seconds.

On the other hand, FIG. 12B illustrates recovery time of a sensor when there is a gas sensor in an apparatus, and it may be confirmed that since apparatus design is not good, the recovery time takes about 18 minutes.

In Step 317, the environmental sensor measuring and correcting system may retrieve the factory setting (or correcting parameter) value stored in the memory and update it to a function required for actual measurement and correction. Accordingly, the present invention may perform sensor correction work of Steps 321 to 324 for every measurement.

In Step 321, the environmental sensor measuring and correcting system may acquire air pollutant concentration value from the environmental sensor. At this time, in case of analog output, the acquiring method measures output voltage and converting it to concentration is performed, and in case of digital output, the acquired value may be used as it is. Afterwards, the environmental sensor measuring and correcting system according to an example of embodiments performs a process for removing noise, and at this time, the process for removing noise uses the noise removing filter and low pass filter. This was described above, so it will be omitted.

Based on air pollutant concentration value using an environment sensor in which noise is removed, error according to concentration may be corrected. More particularly, error according to actual measured air pollutant concentration value may be occurred, and gradient value according to the concentration may change. For example, it is supposed that it is normal that a first fine dust sensor measures 5 ug of fine dust concentration in an environment in which fine dust is 0 ug, and the fine dust sensor measures 55 ug of fine dust concentration in an environment in which fine dust is 50 ug, when a second fine dust sensor measures 10 ug of fine dust concentration in the environment in which fine dust is 0 ug, the second fine dust sensor measures 55 ug or 56 ug of fine dust concentration, not 60 ug of fine dust concentration, in the environment in which fine dust is 50 ug.

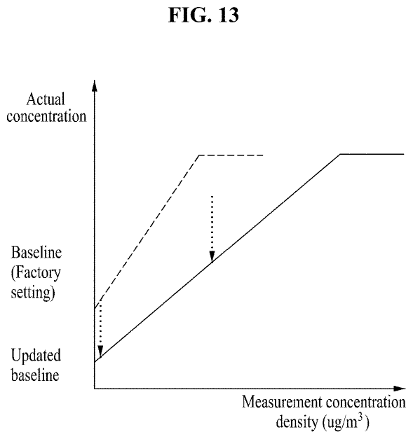

Accordingly, in Step 322, as illustrated in FIG. 13, the environmental sensor measuring and correcting system may correct error according to air pollutant concentration through above described [Equation 1] according to the updated baseline of the environmental sensor in which noise is removed.

Afterwards, in Step 323 and 324, the environmental sensor measuring and correcting system may measure temperature-humidity and correct the environmental sensor value according to the temperature-humidity.

The environmental sensor according to an example of embodiment converts the amount of air pollutant by using an optical method, and for photodiode used in the environmental sensor, when light is incident, reverse current flows in proportion to the amount of incident light and this is measured and converted to amount of dust. Meanwhile, even when there is no incident light in the light receiving element, flowing current is called dark current, and the dark current increases double every 5 degrees or 10 degrees. Also, response characteristics may change depending on wavelength.

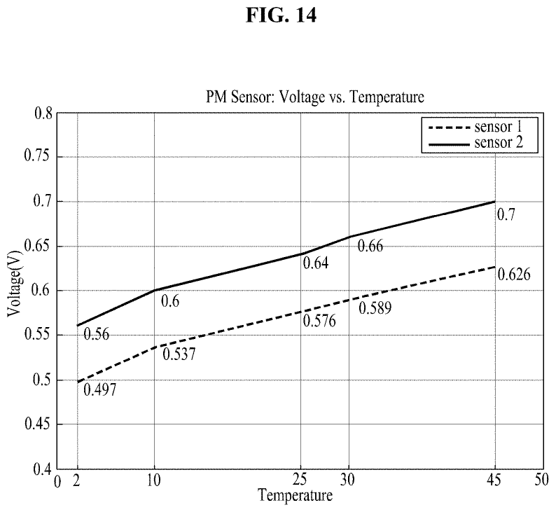

Describing with reference to FIG. 14, FIG. 14 illustrates output values occurred in case of changing temperature as a graph, and it may be confirmed that when temperature changes by one degree, it is converted to about 0.5 ug of fine dust concentration. It may be known that this leads to a result that error is included in find dust measurement, so when temperature changes to 40 degrees, error of fine dust increases by 20 ug.

To correct such error, the environmental sensor measuring and correcting system uses described above [Equation 2], and may store it as lookup table and correct and use temperature value every measurement.

Humidity is also important factor changing characteristics of the environmental sensor. Water vapor causes light scattering of the environmental sensor, which makes it be recognized as air pollutant, and makes various air pollutant particles agglomerate to be displayed with larger and more concentration. Therefore, correction accordingly is needed.

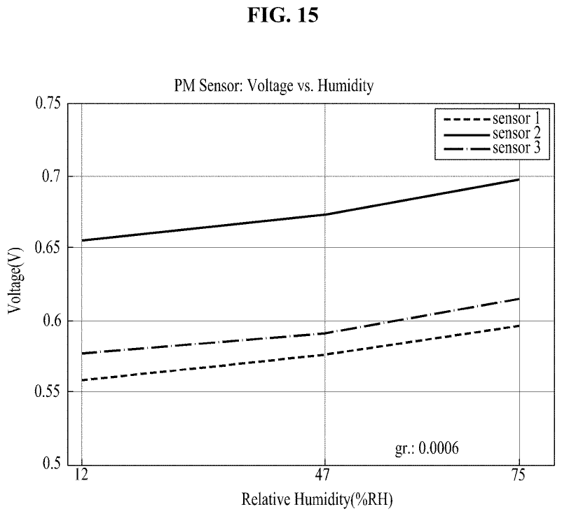

Describing in reference to FIG. 15, FIG. 15 illustrates output values by each humidity as a graph, and it may be confirmed that even when actual fine dust is 0 ug, amount of fine dust increases according to amount of humidity, and it may be known that when there is about 60% RH change, fine dust concentration increases by 6.5 ug.

To correct such error, the environmental sensor measuring and correcting system uses above described [Equation 3], and may store it as lookup table and correct and use humidity value every measurement.

Afterwards, in Step 325, the environmental sensor measuring and correcting system may store the corrected baseline value and the corrected temperature-humidity value of the environmental sensor in the memory or the database unit.

At the same time, in Steps 331 and 332, the environmental sensor measuring and correcting system may classify pollution level for air pollutant concentration value measured by using the corrected environmental sensor and temperature-humidity sensor into 5 steps, e.g., very bad, bad, normal, good, very good, and this may be LED or displayed as pollution status.

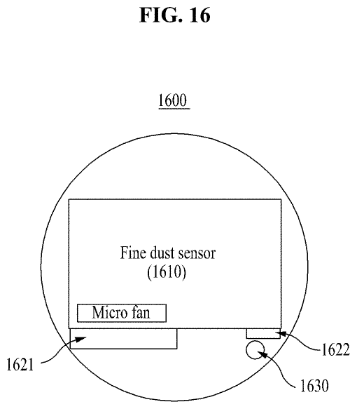

FIG. 16 illustrates an example of structure of an environmental sensor measuring and correcting system according to an example of embodiments.

An environmental sensor measuring and correcting system according to an example of embodiments 1600 needs to have an air circulation system for fast stabilization time and accurate VOC measurement. For this, a simple measurement system having air circulation by using a separate fan such as an outlet 1621 is proposed. The environmental sensor measuring and correcting system 1600 may be designed so that the fan expels air in an apparatus to outside during operation.

According to embodiment examples, the environmental sensor measuring and correcting system 1600 may make air circulation in the apparatus only at the point that VOC is measured through a VOC sensor 1630 for low power operation.

At this time, an independent fan may be used, but since a fine dust sensor 1610 have a micro fan in order to cause convection current, an air circulation system for VOC may be configured by positioning the VOC sensor 1630 in front of an inlet 1622 of the fine dust sensor 1610.

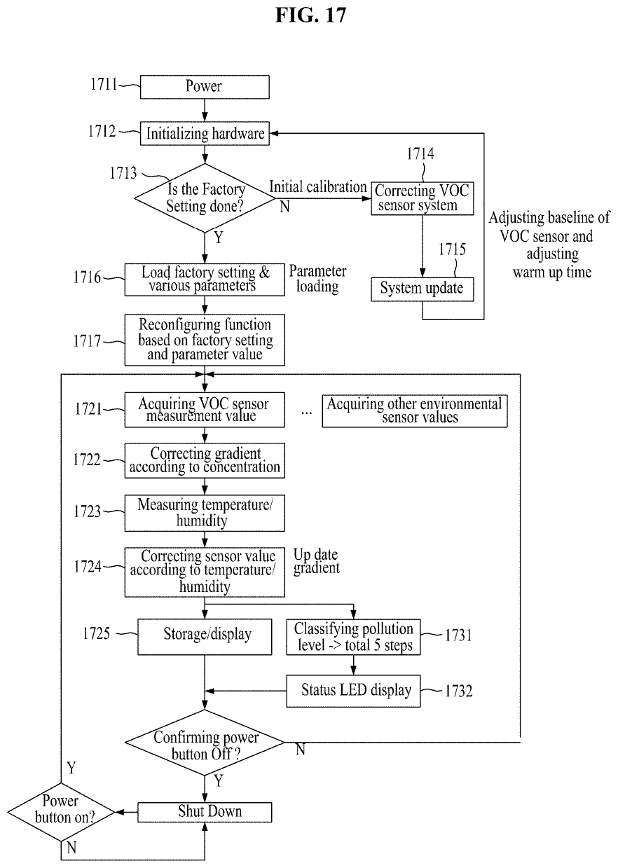

FIG. 17 is a flow chart illustrating an operation method of an environmental sensor measuring and correcting system according to an example of embodiments.

Referring to FIG. 17, an environmental sensor measuring and correcting system according to an example of embodiments may represent a flow chart for VOC sensor measurement and correction as the same with illustrated in FIG. 3. There is a difference in that the VOC sensor is used instead of the environmental sensor of FIG. 3.

Accordingly, since the flow chart was described in detail in FIG. 3, hereinafter, description for the operation method of the environmental sensor measuring and correcting system of FIG. 17 will be omitted.

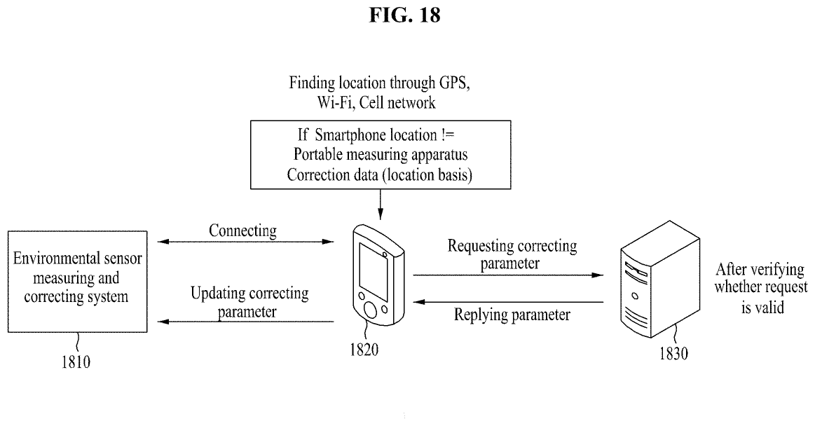

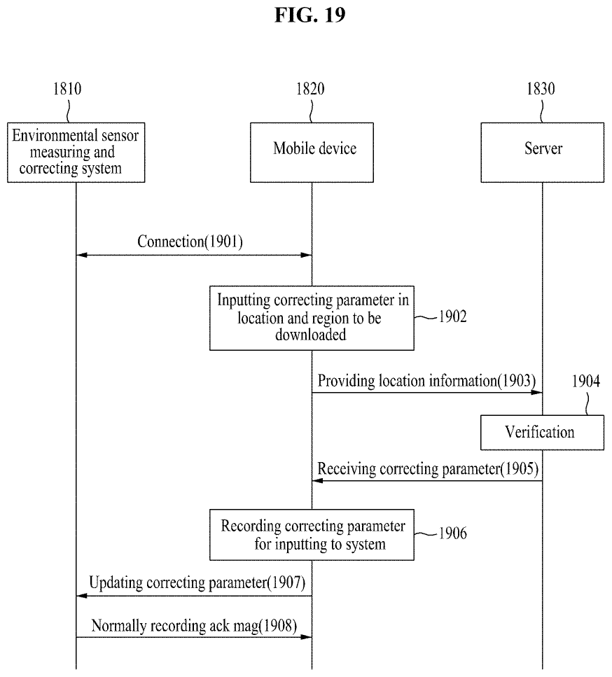

FIGS. 18 and 19 are drawings for illustrating example of updating a correcting parameter according to an example of embodiments.

Referring to FIG. 18, an environmental sensor measuring and correcting system according to an example of embodiments 1810 may be interlocked with a mobile device 1820, and the mobile device 1820 may communicate with an external server 1830. At this time, the environmental sensor measuring and correcting system 1810 may use an environmental sensor of a fine dust sensor, a VOC (Volatile Organic Compound) sensor or a gas sensor.

In case of air pollutant, since they are optically measured, they are differently scattered depending on size and shape of particles, and because of this, even the same size is recognized in different amounts depending on the shape of scattered particles.

Different correcting parameters should be applied according to location and region used for this. However, when a user A who lives in Seoul tries to move to Busan and measure air pollutant concentration, since it is measured with an environmental sensor corrected in Seoul, error on this occurs. In other words, this error is caused by the difference between air pollutant distribution in Seoul and air pollutant distribution in Busan.

For example, in Seoul, it may exist that 10 ug of fine dust concentration is 50%, 5 ug of fine dust concentration is 20%, and 25 ug or less of fine dust concentration is 30% in the air. On the other hand, if it is supposed that it exists that 10 ug of fine dust concentration is 40%, 5 ug of fine dust concentration is 10%, and 25ug or less of fine dust concentration is 50% in Busan, optimal correcting parameter for this distribution may minimize the error in the measurement.

However, the environmental sensor measuring and correcting system 1810, which is a portable fine dust measurement apparatus, is hard to record the correcting parameter for all regions and locations, and also hard to measure the locations. Therefore, when default correcting numerical value is input in the environmental sensor measuring and correcting system 1810 which is a portable fine dust measurement apparatus, the correcting parameter may be updated when location is changed through the mobile device 1820.

For example with reference to FIG. 18, since the correcting parameter of Seoul is input in the environmental sensor measuring and correcting system 1810, if the location of the mobile device 1820 is moved to Busan, it may confirm whether the correcting parameter will be updated since the location is changed through a pop-up to the user. Accordingly, the correcting parameter may be updated according to setting value of update each time it is updated according to the user's confirmation or automatically changed.

More particularly, when the environmental sensor measuring and correcting system 1810 is interlocked with the mobile device 1820, and location of an application of the mobile device 1820 is changed, the mobile device 1820 provides a correcting parameter of region corresponding to the corresponding location information to the external server 1830 and verifies it, and then, provides the returned correcting parameter to the environmental sensor measuring and correcting system 1810, and the environmental sensor measuring and correcting system 1810 may update the changed correcting parameter.

Also, the user may directly input the correcting parameter to the environmental sensor measuring and correcting system 1810 which is a portable fine dust measurement apparatus by using the mobile device 1820.

For example with reference to FIG. 19, in a state that the environmental sensor measuring and correcting system 1810 and the mobile device 1820 are interlocked, the user may input the correcting parameter in various locations and regions to be downloaded (Steps 1901 and 1902).

Accordingly, through Steps 1903 to 1905, the mobile device 1820 may receive the verified correcting parameter from the external server 1830 by providing fine dust distribution information for corresponding location and region.

In Step 1906, the mobile device 1820 records the received correcting parameter from the external server 1830 and provides it to the environmental sensor measuring and correcting system 1810, and the environmental sensor measuring and correcting system 1810 may update the verified correcting parameter, and then, transmit ACK signal to the mobile device 1820 (Steps 1907 and 1908).

In other words, when the location of the mobile device 1820 is different from the correcting parameter of the environmental sensor measuring and correcting system 1810, the user may directly input the correcting parameter according to the location change through the mobile device 1820, and the environmental sensor measuring and correcting system 1810 may update the verified parameter through the external server 1830 after being input by the user.

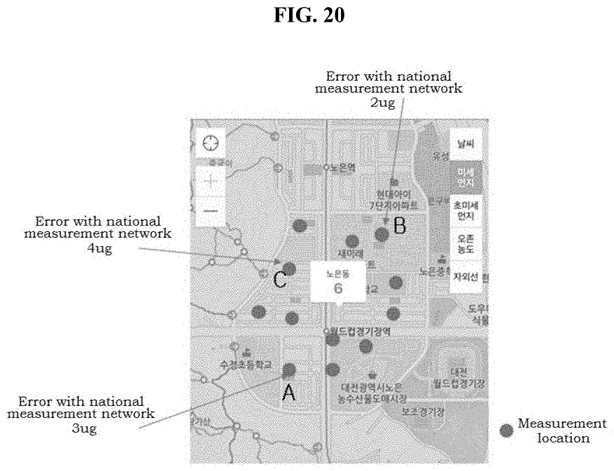

FIG. 20 is a drawing for illustrating an example of correcting baseline by using national measurement network data according to an example of embodiments.

Reference of the present invention uses national measurement network data. An environmental sensor measuring and correcting system according to an example of embodiments may find near national measurement network based on location information acquired from a mobile device. At this time, since error may increase if distance between the measurement network and the mobile device is far, the environmental sensor measuring and correcting system corrects baseline according to drift within preset radius, e.g., when there is 200 m measurement network.

For example, in the present invention, when the measured fine dust data is 55 ug and data of nearby national measurement network device indicates 50 ug, 5 ug error occurs. The environmental sensor measuring and correcting system determines the occurred difference value as error for baseline, and may correct baseline offset of an environment sensor. However, if only one-time or short time data is collected, errors on the measurement may be included. Accordingly, the present invention may find optimal value by collecting a lot of data, and then, correct the offset.

Describing a process for using national measurement network data, the environmental sensor measuring and correcting system may measure air pollutant by using the environmental sensor and store the data in a database unit or a mobile device.

At this time, the environmental sensor measuring and correcting system tries to update baseline when baseline update of the environmental sensor is periodically needed, or there is error with nearby national measurement network data, error exceeds a certain threshold, or error exceeds a certain threshold for more than 10 days in a row.

Describing in detail, as illustrated in FIG. 20, the environmental sensor measuring and correcting system may select a national measurement network device which likely to obtain the best data in the current location (multiple selection is possible) or select a national measurement network device including the most data within a preset radius, calculate difference between all measurement data within the preset radius and national measurement network measurement apparatus based on the selected national measurement network device, obtain offset with their average value, and update baseline of the environmental sensor according to the offset.

As another example, the environmental sensor measuring and correcting system may calculate more accurate offset by using kNN (k-nearest neighbor) or artificial intelligence, and update baseline of the environmental sensor according to the offset.

The environmental sensor measuring and correcting system may update baseline offset for the environmental sensor through interlocked with the mobile device, and at this time, may ask the user whether update or not through the mobile device.

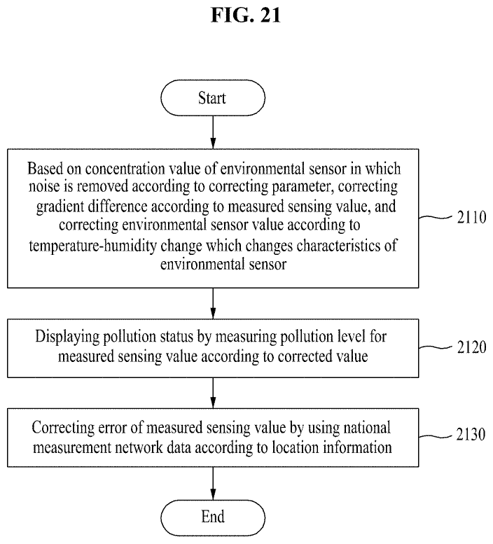

FIG. 21 is a flow chart illustrating an operation method of an environmental sensor measuring and correcting method according to an example of embodiments.

A method of FIG. 21 is performed by the environmental sensor measuring and correcting system illustrated in FIG. 1.

Referring to FIG. 21, in Step 2110, noise of an environmental sensor is removed, and based on air pollutant concentration value of the environmental sensor in which noise is removed according to a correcting parameter, gradient difference according to measured sensing value is corrected, and environmental sensor value according to temperature-humidity change which changes characteristics of the environmental sensor is corrected.

Step 2110 may acquire baseline by removing the noise of the environmental sensor by applying a noise removing filter, and adjust baseline by adjusting the correcting parameter according to gradient difference value of the environmental sensor acquired in a first environment and a second environment having different measurement concentrations.

For example, Step 2110 may remove the noise for the environmental sensor by using the noise removing filter, and then, acquire baseline corresponding to the corresponding environmental sensor. Since environmental sensors have different initial baseline value recognized for each sensor, Step 2110 may first perform acquiring baseline for the corresponding environmental sensor in the system.

Afterwards, Step 2110 may correct gradient difference according to measured sensing value based on the adjusted baseline according to the correcting parameter on the system.

Afterwards, Step 2110 may correct temperature-humidity value of the environmental sensor by using the measured sensing value, difference value between reference temperature and the current temperature, and difference value of reference humidity and the current humidity.

In Step 2120, according to the corrected value, pollution level for the measured sensing value is measured and pollution status is displayed.

For example, Step 2120 may classify pollution level into about 5 steps according to the measured sensing value, the pollution level is measured through the classification by step from low risk to high risk, and pollution level of the current air pollutant may be displayed through at least one of LED, display, and output sound.

In Step 2130, error of the measured sensing value is corrected by using national measurement network data according to location information.

Step 2130 may correct error of the measured sensing value by adjusting baseline of the environmental sensor according to national measurement network data according to location information acquired through the interlocked mobile device.

Also, Step 2130 may correct error of the measured sensing value by adjusting baseline of the environmental sensor by drift caused by hours of use.

Also, the environmental sensor measuring and correcting method may further include updating the correcting parameter by using environmental distribution information in the current location and region according to location information (not illustrated).

The updating the correcting parameter may receive and update the correcting parameter for environmental distribution information in the current location and region verified in an external server by location information acquired through the interlocked mobile device from the mobile device, and adjust baseline of the environmental sensor according to the updated correcting parameter.

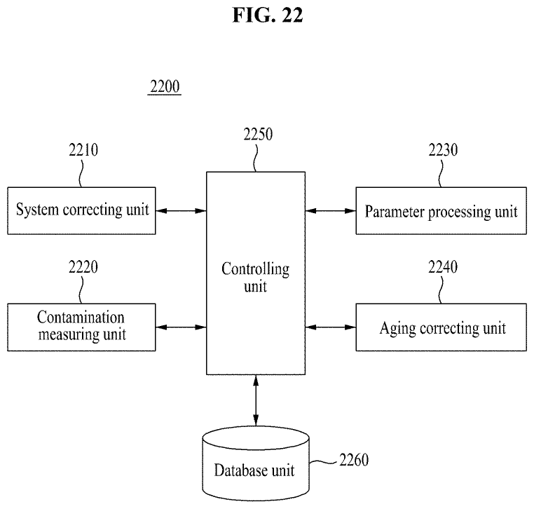

FIG. 22 is a block diagram illustrating detailed configuration of an environmental sensor measuring and correcting system according to an example of embodiments.

Referring to FIG. 22, an environmental sensor measuring and correcting system according to an example of embodiments measures harmful environmental pollution level by using a fine dust sensor in which data is corrected.

For this, an environmental sensor measuring and correcting system according to an example of embodiments 2200 may include a system correcting unit 2210 and a contamination measuring unit 2220, and may further include a parameter processing unit 2230, an aging correcting unit 2240, a controlling unit 2250, and a database unit 2260.

The environmental sensor measuring and correcting system 2200, which is to measure more accurate fine dust pollution level by correcting data of a fine dust sensor, may be in a form in which the fine dust sensor is included in structure of the system.

At this time, although it is described that the environmental sensor measuring and correcting system 2200 uses the fine dust sensor, besides the fine dust sensor, an air pollution measurement sensor may also be applicable.

The system correcting unit 2210 may remove noise of the fine dust sensor, and based on fine dust concentration value of the fine dust sensor in which noise is removed according to a correcting parameter, may correct gradient difference according to the measured fine dust concentration value, and correct fine dust sensor value according to temperature-humidity change which changes fine dust characteristics.

In initial stage of the system, the system correcting unit 2210 may acquire baseline by removing noise of the fine dust sensor by applying a noise removing filter, and adjust baseline by adjusting the correcting parameter according to the gradient difference according to the fine dust concentration acquired in a first environment and a second environment having different fine dust concentrations.

For example, the system correcting unit 2210 may remove noise for the environmental sensor by using the noise removing filter, and then, acquire baseline corresponding to the corresponding fine dust sensor. Since the fine dust sensor has different initial baseline value recognized for each sensor, the system correcting unit 2210 may first perform acquiring baseline for the corresponding fine dust sensor in the system.

Furthermore, since the fine dust sensors have different baselines, this should be corrected in an environment in which there is no fine dust. Accordingly, the system correcting unit 2210 measures fine dust concentration in each of a first environment in which fine dust is 0 ug and a second environment in which fine dust is about 50 ug, and infers offset difference changed according to the fine dust concentration by adjusting gradient value according to the fine dust concentration value, and because of this, baseline may be adjusted by adjusting the correcting parameter of the fine dust sensor.

In next stage of the system, the system correcting unit 2210 may correct gradient difference according to measured fine dust concentration value based on baseline adjusted according to the correcting parameter on the system.

The environmental sensor measuring and correcting system 2200 acquires fine dust concentration from the fine dust sensor. At this time, in case of analog method, the acquiring method measures output voltage and converting it to concentration is performed, and in case of digital output, the acquired value is used as it is. Afterwards, the present invention performs a process for removing noise, and the process for removing noise may be performed by applying the noise filter as the same with the operation performed in the initial stage of the system.

Accordingly, the system correcting unit 2210 may correct gradient difference according to fine dust concentration value measured based on baseline by using above described [Equation 1] in order to correct error according to concentration based on fine dust concentration of the fine dust sensor in which noise is removed.

Afterwards, the system correcting unit 2210 may correct temperature-humidity value of the fine dust sensor by using the measured fine dust concentration value, difference value of reference temperature and the current temperature, and difference value of reference humidity and the current humidity.

When measuring fine dust, the temperature-humidity value may affect fine dust concentration value. Since error occurs on fine dust measurement by causing change on the fine dust concentration value by the temperature-humidity value, correction of the temperature-humidity is needed when measuring fine dust. Accordingly, the system correcting unit 2210 may correct the temperature-humidity value of the fine dust sensor by using difference value of reference temperature and the current temperature and difference value of reference humidity and the current humidity through above described [Equation 2] and [Equation 3].

The contamination measuring unit 2220 displays pollution status by measuring pollution level for the measured fine dust concentration value according to the corrected value.

For example, the contamination measuring unit 2220 may classify pollution level into about 5 steps according to the measured fine dust concentration value, the pollution level is measured through the classification by step from low risk to high risk, and pollution status of the current fine dust may be displayed through at least one of LED, display, and output sound.

The parameter processing unit 2230 may update the correcting parameter by using fine dust distribution information in the current location and region according to location information.

The environmental sensor measuring and correcting system 2200 may further include a communication module (not illustrated), and may be interlocked with a mobile device that a user has through the communication module. At this time, the mobile device may be at least one of a smartphone, a desktop, a PC, a mobile terminal, a PDA, a laptop, a tablet PC, and a wearable device, and may be installed with an application for interworking with the environmental sensor measuring and correcting system 100. Furthermore, the mobile device may receive selection input of a user, and since it may include a display in a form of a touch screen which may perform a predetermined set of functions through a screen including touch-sensing area or may be a device including at least one physical button or virtual button, the kinds and forms are not limited thereto.

Accordingly, the environmental sensor measuring and correcting system 2200 may receive location information in real-time through the mobile device, and update the correcting parameter by using fine dust distribution information in the current location and region according to location information. Particularly, the parameter processing unit 2230 may receive and update the correcting parameter for the fine dust distribution information in the current location and region verified in an external server by location information acquired through the interlocked mobile device from the mobile device, and adjust baseline of the fine dust sensor according to the updated correcting parameter.

For example, when a user A who lives in Seoul moves to Busan and measures fine dust concentration, since it is measured with a fine dust sensor corrected in Seoul, error may occurs according to difference in fine dust distribution in Seoul and fine dust distribution in Busan. Accordingly, the parameter processing unit 2230 may adjust baseline of the fine dust sensor by updating the correcting parameter for fine dust distribution information for Busan.

The aging correcting unit 2240 may correct the measured fine dust concentration's error occurred according hours of use.

In case of the fine dust sensor, since dust builds up inside over time, and foreign substance changes measured value of the optical device, measurement error may occur by aging over time. At this time, baseline correction is needed rather than overall correction.

Accordingly, the aging correcting unit 2240 may correct error of the measured fine dust concentration value by adjusting baseline of the fine dust sensor by drift caused by hours of use. For example, the aging correcting unit 2240 may correct offset so that baseline value of the fine dust sensor is displayed as 0 ug in an environment in which fine dust is 0 ug.

The controlling unit 2250 may control operation of the system correcting unit 2210, the contamination measuring unit 2220, the parameter processing unit 2230, and the aging correcting unit 2240 of the environmental sensor measuring and correcting system 2200, and may store data occurred in each component in the database unit 2260.

The database unit 2260 may store baseline value of the fine dust sensor corrected in the fine dust sensor correcting process, and store and maintain the correcting value for gradient value and measured temperature and humidity according to concentration value measured with the fine dust sensor.

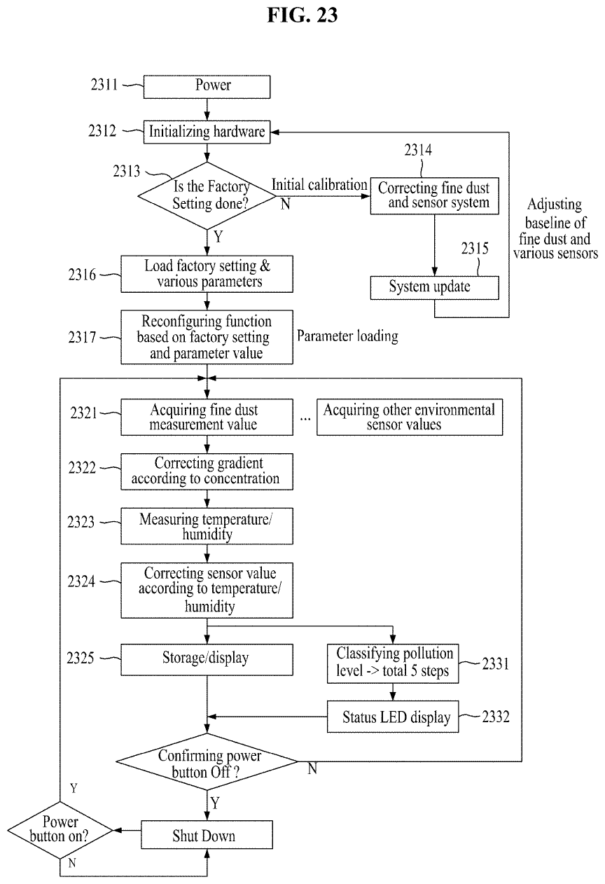

FIG. 23 is a flow chart illustrating an operation method of an environmental sensor measuring and correcting system according to an example of embodiments.

Referring to FIG. 23, an environmental sensor measuring and correcting system according to an example of embodiments confirms whether correction of fine dust sensors is performed by confirming factory setting (or correcting parameter) according to hardware initialization when initial power is applied (Steps 2311 to 2313). Processes of Steps 2311 to 2313 are performed only in initial stage of powering up the environmental sensor measuring and correcting system.

The fine dust sensor has different baseline value recognized for each sensor. For example, when find dust is 10 ug/m³, a first fine dust sensor may measure fine dust concentration as 15 ug, and a second fine dust sensor may measure and display fine dust concentration as Bug. Accordingly, the environmental sensor measuring and correcting system measures and corrects baseline of each different environmental sensor through Steps 2311 to 2317, and stores it in a system memory (or a database unit).

A graph illustrated in FIG. 4 may indicate a result value when measuring through 7 fine dust sensors and removing noise, and it may be confirmed that they represent all different baselines. Also, a graph illustrated in FIG. 5 indicates a measurement value measured through 7 fine dust sensors, and it may be confirmed that a lot of noise is included in the measured fine dust concentration value.

Accordingly, the environmental sensor measuring and correcting system may correct baseline by removing noise of the fine dust sensor in Steps 2314 and 2315.

The environmental sensor measuring and correcting system may remove noise of the fine dust sensor by using the noise removing filter of FIG. 6 and above described [Equation 4]. The result is displayed every one second in FIG. 6, and this value uses weighted moving average of whole frame. Also, window size is determined between 15 to 30, and may be differently applied according to the environment to be measured.

When the noise filter is applied, the environmental sensor measuring and correcting system may extract signal as illustrated in FIG. 7. However, each of 7 fine dust sensors has different baseline in FIG. 7, this should be corrected in an environment in which there is no fine dust.

Accordingly, in Steps 2314 and 2315, the environmental sensor measuring and correcting system may acquire concentration value of the fine dust sensor in an environment in which fine dust is 0 ug and record it in a memory, and update baseline of the corrected fine dust sensor in an environment in which there is no fine dust. For example, when fine dust concentration is measured as 5 ug in an environment in which fine dust is 0 ug, since offset is 5 ug, subtract this value every measurement.

Referring to FIGS. 8 and 9, since each of 7 fine dust sensors has different baseline value, error according to actual measured fine dust concentration occurs. In other words, gradient value according to fine dust concentration may be different. For example, if it is supposed that it is normal that in an environment in which fine dust is 0 ug, a first fine dust sensor measures 5 ug of fine dust concentration, and in an environment in which fine dust is 50 ug, the first find dust sensor measures 55 ug of fine dust concentration, in case that a second fine dust sensor measures 10 ug of fine dust concentration in the environment in which fine dust is 50 ug, the second fine dust sensor measures 55 ug or 56 ug of fine dust concentration, not 60 ug of fine dust concentration in the environment in which fine dust is 50 ug.

This is caused by photodiode (light receiving unit) of the fine dust sensor and analogue circuit, and when the circuit of the fine dust sensor works with 3V, signal may be acquired by 3V output. In other words, this is a problem which occurs as upper boundary is fixed and lower boundary is variable.

For this, the environmental sensor measuring and correcting system should perform correction according to fine dust concentration, and this may be corrected by acquiring and adjusting gradient value in a linear function.

In initial factory calibration, the environmental sensor measuring and correcting system may be input with fine dust concentration value acquired through at least two fine dust sensors. For example, the environmental sensor measuring and correcting system may acquire fine dust concentration value in an environment in which fine dust concentration is 0 ug, and obtain gradient value by acquiring second fine dust concentration value in an environment in which fine dust concentration is 100 ug. Accordingly, it is possible to infer offset difference which changes according to fine dust concentration.

In Step 2317, the environmental sensor measuring and correcting system may retrieve factory setting (or correcting parameter) stored in the memory and update it as a function needed for actual measurement and correction. Accordingly, the environmental sensor measuring and correcting system may perform sensor correcting work of Steps 2321 to 2324 for every measurement value.

In Step 2321, the environmental sensor measuring and correcting system may acquire fine dust concentration value from the fine dust sensor. At this time, in case of analog output, the acquiring method measures output voltage and converting it to concentration is performed, and in case of digital output, the acquired value may be used as it is. Afterwards, the environmental sensor measuring and correcting system performs a process for removing noise, and at this time, the process for removing noise uses the noise removing filter and low pass filter. This was described above, so it will be omitted.

Based on fine dust concentration value using the environment sensor in which noise is removed, error according to the fine dust concentration may be corrected. More particularly, error according to actual measured fine dust concentration value may be occurred, and gradient value according to the concentration may change. For example, if it is supposed that it is normal that a first fine dust sensor measures 5 ug of fine dust concentration in an environment in which fine dust is 0 ug, and the first fine dust sensor measures 55 ug of fine dust concentration in an environment in which fine dust is 50 ug, when a second fine dust sensor measures 10 ug of fine dust concentration in the environment in which fine dust is 0 ug, the second fine dust sensor measures 55 ug or 56 ug of fine dust concentration, not 60 ug of fine dust concentration, in the environment in which fine dust is 50 ug.

Accordingly, in Step 2322, the environmental sensor measuring and correcting system may correct error according to the fine dust concentration through above described [Equation 1] according to the updated baseline of the fine dust sensor in which noise is removed as illustrated in FIG. 13.

Afterwards, in Steps 2323 and 2324, the environmental sensor measuring and correcting system may correct fine dust sensor value according to temperature-humidity by measuring temperature-humidity.

The environmental sensor according to an example of embodiments converts the amount of fine dust by using an optical method, and for photodiode used in the environmental sensor, when light is incident, reverse current flows in proportion to the amount of incident light and this is measured and converted to amount of dust. Meanwhile, even when there is no incident light in the light receiving element, flowing current is called dark current, and the dark current increases double every 5 degrees or 10 degrees. Also, response characteristics may change depending on wavelength.

Describing with reference to FIG. 14, FIG. 14 illustrates output values occurred in case of changing temperature as a graph, and it may be confirmed that when temperature changes by one degree, it is converted to about 0.5 ug of fine dust concentration. It may be known that this leads to a result that error is included in find dust measurement, so when temperature changes to 40 degrees, error of fine dust increases by 20 ug.

To correct such error, the environmental sensor measuring and correcting system uses above described [Equation 2], and may store it as lookup table and correct and use temperature value every measurement.

Humidity is also important factor changing characteristics of the fine dust sensor. Water vapor causes light scattering of the fine dust sensor, which makes it be recognized as fine dust, and makes various fine dust particles agglomerate to be displayed with larger and more concentration. Therefore, correction accordingly is needed.

Describing with reference to FIG. 15, FIG. 15 illustrates output values by each humidity as a graph, and it may be confirmed that even when actual fine dust is 0 ug, amount of fine dust increases according to amount of humidity, and it may be known that when there is about 60% RH change, fine dust concentration increases by 6.5 ug.

To correct such error, the environmental sensor measuring and correcting system uses above described [Equation 3], and may store it as lookup table and correct and use humidity value every measurement.

Afterwards, in Step 2325, the environmental sensor measuring and correcting system may store the corrected baseline value and the corrected temperature-humidity value of the fine dust sensor in the memory or the database unit.

At the same time, in Steps 2331 and 2332, the environmental sensor measuring and correcting system may classify pollution level for fine dust concentration value measured by using the corrected fine dust sensor and temperature-humidity sensor into 5 steps, e.g., very bad, bad, normal, good, very good, and this may be LED or displayed as pollution status.

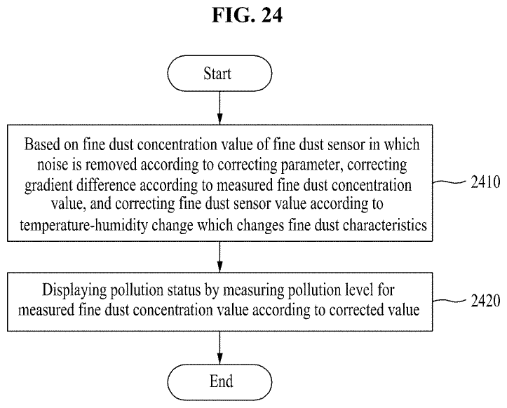

FIG. 24 is a flow chart illustrating an operation of an environmental sensor measuring and correcting method according to an example of embodiments.

A method of FIG. 24 is performed by the environmental sensor measuring and correcting system illustrated in FIG. 22.

Referring to FIG. 24, in Step 2410, noise of a fine dust sensor is removed, and based on fine dust concentration value of the fine dust sensor in which noise is removed, gradient difference according to measured fine dust concentration value is corrected, and fine dust sensor value according to temperature-humidity change which changes fine dust characteristics is corrected.

Step 2410 may acquire baseline by removing noise of the fine dust sensor by applying a noise removing filter, and adjust baseline by adjusting a correcting parameter according to gradient value according to fine dust concentration acquired in a first environment and a second environment having different fine dust concentrations.

For example, Step 2410 may remove noise for the fine dust sensor by using the noise removing filter, and then, acquire baseline corresponding to the corresponding fine dust sensor. Since fine dust sensor have different initial baseline value recognized for each sensor, Step 2410 may first perform acquiring baseline for the corresponding fine dust sensor in the system.

Afterwards, Step 2410 may correct gradient difference according to measured fine dust concentration value based on the adjusted baseline according to the correcting parameter on the system.

Afterwards, Step 2410 may correct temperature-humidity value of the fine dust sensor by using the measured fine dust concentration value, difference value between reference temperature and the current temperature, and difference value of reference humidity and the current humidity.

In Step 2420, according to the corrected value, pollution level for the measured fine dust concentration is measured and pollution status is displayed.

Step 2420 may classify pollution level into about 5 steps according to the measured fine dust concentration value, the pollution level is measured through the classification by step from low risk to high risk, and pollution level of the current fine dust may be displayed through at least one of LED, display, and output sound.

Also, the environmental sensor measuring and correcting method may further include a first step updating the correcting parameter by using fine dust distribution information in the current location and region according to location information (not illustrated) and a second step correcting error of the measured fine dust concentration value occurred by hours of use (not illustrated).

The first step may receive and update the correcting parameter for the fine dust distribution information in the current location and region verified from an external server by location information acquired through an interlocked mobile device from the mobile device, and adjust baseline of the fine dust sensor according to the updated correcting parameter.

The second step may correct error of the measured fine dust concentration by adjusting baseline of the fine dust sensor by drift caused by hours of use.

The units described herein may be implemented using hardware components, software components, and/or a combination thereof. For example, a processing device may be implemented using one or more general-purpose or special purpose computers, such as, for example, a processor, a controller, an ALU (arithmetic logic unit), a digital signal processor, a microcomputer, a FPGA (field programmable gate array), a PLU (programmable logic unit), a microprocessor or any other device capable of responding to and executing instructions in a defined manner. The processing device may run an operating system (OS) and one or more software applications that run on the OS. The processing device also may access, store, manipulate, process, and create data in response to execution of the software. For purpose of simplicity, the description of a processing device is used as singular; however, one skilled in the art will be appreciated that a processing device may include multiple processing elements and multiple types of processing elements. For example, a processing device may include multiple processors or a processor and a controller. In addition, different processing configurations are possible, such as parallel processors.

The software may include a computer program, a piece of code, an instruction, or some combination thereof, for independently or collectively instructing or configuring the processing device to operate as desired. Software and/or data may be embodied permanently or temporarily in any type of machine, component, physical or virtual equipment, computer storage medium or device or propagated signal wave to provide instructions or data to or be interpreted by the processing device. The software also may be distributed over network coupled computer systems so that the software is stored and executed in a distributed fashion. In particular, the software and data may be stored by one or more computer readable recording mediums.

The method according to the example embodiments may be implemented in a form of program instruction which may be performed through various computer means and recorded in computer-readable media. The media may also include, alone or in combination with the program instructions, data files, data structures, and the like. The program instructions stored in the media may be specially designed and constructed for the present invention or they may be of well-known and available to those having skill in the computer software arts. Examples of the media include magnetic media such as hard disks, floppy disks, and magnetic tape; optical media such as CD-ROM disks and DVD; magneto-optical media such as floptical disks; and hardware devices that are specially configured to store and perform program instructions, such as ROM (read-only memory), RAM (random access memory), flash memory, and the like. Examples of program instructions include both machine code, such as produced by a compiler, and higher level code that may be executed by the computer using an interpreter. The hardware apparatus may be configured to operate one or more software modules in order to perform an operation of an embodiment, and vice versa.

While certain example embodiments and implementations have been described herein, other embodiments and modifications will be apparent from this description. Accordingly, the invention is not limited to such embodiments, but rather to the broader scope of the presented claims and various obvious modifications and equivalent arrangements.

What is claimed is:

1. An environmental sensor measuring and correcting system comprising:
a system correcting unit for removing noise of an environmental sensor, correcting gradient difference according to measured sensing value based on air pollutant concentration value of the environmental sensor in which the noise is removed according to a correcting parameter, and correcting environmental sensor value according to temperature-humidity change which changes characteristics of the environmental sensor;
a contamination measuring unit for displaying pollution status by measuring pollution level for the measured sensing value according to corrected value; and
a baseline correcting unit for correcting error of the measured sensing value by using national measurement network data based on location information.

2. The environmental sensor measuring and correcting system of claim 1, wherein the environmental sensor is a fine dust sensor, a VOC (Volatile Organic Compound) sensor, or a gas sensor.

3. The environmental sensor measuring and correcting system of claim 1, wherein the system correcting unit acquires baseline by removing noise of the environmental sensor by applying a noise removing filter in the initial stage, and adjusts the baseline by adjusting the correcting parameter according to gradient difference of the environmental sensor acquired in a first environment and a second environment having different measurement concentrations.

4. The environmental sensor measuring and correcting system of claim 3, wherein the system correcting unit corrects gradient difference according to the measured sensing value based on the adjusted baseline according to the correcting parameter on the system.

5. The environmental sensor measuring and correcting system of claim 4, wherein the system correcting unit corrects temperature-humidity value of the environmental sensor by using the measured sensing value, difference value of reference temperature and the current temperature, and difference value of reference humidity and the current humidity.

6. The environmental sensor measuring and correcting system of claim 1 further comprising a parameter processing unit for updating the correcting parameter by using environmental distribution information in the current location and region according to the location information.

7. The environmental sensor measuring and correcting system of claim 6, wherein the parameter processing unit receives and updates the correcting parameter for the environmental distribution information in the current location and region verified in an external server by the location information acquired through interlocked mobile device from the mobile device, and adjusts the baseline of the environmental sensor according to the updated correcting parameter.

8. The environmental sensor measuring and correcting system of claim 1, wherein the baseline correcting unit corrects error of the measured sensing value by adjusting the baseline of the environmental sensor according to the national measurement network data according to the location information acquired through interlocked mobile device.

9. The environmental sensor measuring and correcting system of claim 1, wherein the baseline correcting unit corrects error of the measured sensing value by adjusting the baseline of the environmental sensor by drift caused by hours of use.

10. An environmental sensor measuring and correcting system comprising:
a system correcting unit for removing noise of a fine dust sensor, correcting gradient difference according to measured fine dust concentration value based on fine dust concentration value of the fine dust sensor in which the noise is removed according to a correcting parameter, and correcting fine dust sensor value according to temperature-humidity change which changes characteristics of fine dust; and
a contamination measuring unit for displaying pollution status by measuring pollution level for the measured fine dust concentration value according to corrected value.

11. The environmental sensor measuring and correcting system of claim 10, wherein the system correcting unit acquires baseline by removing noise of the fine dust sensor by applying a noise removing filter in the initial stage, and adjusts the baseline by adjusting the correcting parameter according to gradient value according to fine dust concentration acquired in a first environment and a second environment having different fine dust concentrations.

12. The environmental sensor measuring and correcting system of claim 11, wherein the system correcting unit corrects gradient difference according to the measured fine dust concentration value based on the adjusted baseline according to the correcting parameter on the system.

13. The environmental sensor measuring and correcting system of claim 12, wherein the system correcting unit corrects temperature-humidity value of the fine dust sensor by using the measured fine dust concentration value, difference value of reference temperature and the current temperature, and difference value of reference humidity and the current humidity.

14. The environmental sensor measuring and correcting system of claim 10 further comprising a parameter processing unit for updating the correcting parameter by using fine dust distribution information in the current location and region according to location information.

15. The environmental sensor measuring and correcting system of claim 14, wherein the parameter processing unit receives and updates the correcting parameter for the fine dust distribution information in the current location and region verified in an external server by the location information acquired through interlocked mobile device from the mobile device, and adjusts the baseline of the fine dust sensor according to the updated correcting parameter.

16. The environmental sensor measuring and correcting system of claim 10 further comprising an aging correcting unit for correcting error of the measured fine dust concentration value caused by hours of use.

17. The environmental sensor measuring and correcting system of claim 16, wherein the aging correcting unit corrects error of the measured fine dust concentration value by adjusting the baseline of the fine dust sensor by drift caused by hours of use.

18. An environmental sensor measuring and correcting method comprising:
removing noise of a fine dust sensor, correcting gradient difference according to measured fine dust concentration value based on fine dust concentration value of the fine dust sensor in which the noise is removed according to a correcting parameter, and correcting fine dust sensor value according to temperature-humidity change which changes characteristics of fine dust; and
displaying pollution status by measuring pollution level for the measured fine dust concentration value according to corrected value.

19. The environmental sensor measuring and correcting method of claim 18 further comprising updating the correcting parameter by using fine dust distribution information in the current location and region according to location information.

20. The environmental sensor measuring and correcting method of claim 19 further comprising correcting error of the measured fine dust concentration value caused by hours of use.
